(19) United States
(12) Patent Application Publication
(10) Pub. No.: US 2020/0010806 A1
(43) Pub. Date: Jan. 9, 2020

(54) COMPOSITIONS AND METHODS COMPRISING CO-CULTURE OF HEPATOCYTES

(71) Applicant: PhoenixSongs Biologicals, Inc., Branford, CT (US)

(72) Inventors: Marsha Lynn Roach, Branford, CT (US); Richard Harold Malavarca, Long Valley, NJ (US); Shiloh Jesse Barfield, Cary, NC (US)

(73) Assignee: PhoenixSongs Biologicals, Inc., Branford, CT (US)

(21) Appl. No.: 16/482,666

(22) PCT Filed: Jan. 24, 2018
(86) PCT No.: PCT/US2018/015088

§ 371 Date: Jul. 31, 2019

Related U.S. Application Data
(60) Provisional application No. 62/453,020, filed on Feb. 1, 2017.

(51) Int. Cl. C12N 5/071

(52) U.S. Cl. CPC C12N 5/0671; C12N 2502/14; C12N 2513/00; C12N 2501/11; C12N 2533/54

(57) ABSTRACT

Provided are methods and compositions for co-culturing hepatocytes in the presence of growth-arrested stromal cells, resulting in maintenance of hepatocyte function and phenotype for an extended period of time in culture comprising at least 30 days.

### TECHNICAL FIELD

The invention relates to an in vitro co-culture system that combines mammalian primary hepatocytes with mammalian liver non-parenchymal stromal cells which are growth-arrested in amounts that supports or maintains longevity and functionality of isolated hepatocytes, and methods of using the co-culture system for determining the activity or effects of an agent on cells in the cell co-culture model system.

### BACKGROUND ART

Metabolism occurs throughout the body but is concentrated most in the mammalian liver, predominately by epithelial parenchymal cells called hepatocytes. The pharmaceutical industry has historically used both animal and human hepatocytes alone in suspension or monolayer culture as the gold standard for drug metabolism and pharmacokinetics (DMPK), including absorption, distribution, metabolism, excretion and toxicity (ADMET) assays and infectivity of viruses for virology. More recently, co-cultures of supporting cells (feeder cells) of animal origin and human hepatocytes have been developed. While this is the current state of the art in the industry, it is not optimal because hepatotoxicity remains the most frequent cause of the withdrawal of a drug in late stage clinical trials. Thus, there is a need to have in vitro models that better support prediction of hepatotoxicity prior to a drug entering clinical trials. Each withdrawal translates into hundreds of millions or nearly a billion dollars in losses to the pharmaceutical industry. This is primarily due to the fact that currently available assays carried out with primary animal or human hepatocytes are not always predictive of a compound's performance in human subjects.

Hepatocytes constitute approximately 80% of the liver mass and are isolated from donated livers rejected from transplantation or from surgical cut down, also referred to as resections. The livers are subjected to enzymatic digestion followed by separation of the hepatocytes from the non-parenchymal stromal cells to obtain a relatively pure population of hepatocytes. The stromal cells if left with the hepatocytes will overgrow the cultures resulting in death of the hepatocytes. Thus, stromal cells remaining after isolation of the hepatocytes have historically been discarded. Since liver function declines with age, there is a limited resource of mature functional high quality hepatocytes. The limitation of using cryopreserved hepatocytes in monolayer culture is that on average only 10-15% of all livers processed result in isolated hepatocytes that are plateable to the point where the cells can maintain themselves in culture for over 10 days. This forces many studies to be conducted with cells in suspension or in culture with lack of optimal functionality. Hepatocyte longevity in culture is normally only 3-7 days for the best plateable lots, 1-2 days for marginal plateable lots, several days longer if there is an extracellular matrix overlay, and same day assays for the non-plateable suspension lots. While this is the gold standard in the industry, it falls short of what is needed, which is why the FDA launched the initiative to find better in vitro models for hepatocyte maintenance and function. Other co-cultures involving hepatocytes typically have drawbacks (e.g., cultured hepatocytes have one or more of limited viability, and suboptimal function) that limit their wide application for use in modeling one or more of hepatocyte function, and hepatotoxicity.

### SUMMARY OF THE INVENTION

Presented herein is an in vitro co-culture system in which one or more of hepatocyte function and phenotype are maintained in an in vitro culture for an extended period of time. This co-culture system may be used for determining activity or effect (“activity”) of an agent on cells in the co-culture system. In one aspect, determined is activity of an agent on hepatocytes in the co-culture system.

In another aspect, provided is an in vitro co-culture system comprising feeder cells and hepatocytes, wherein the feeder cells and hepatocytes are allogeneic (e.g., the feeder cells and hepatocytes are isolated from the same species), and the feeder cells comprise growth-arrested cells.

In another aspect, provided is an in vitro co-culture system comprising feeder cells comprising stromal cells, and hepatocytes, wherein the feeder cells and hepatocytes are allogenic, and wherein the feeder cells comprise growth arrested cells. In a further aspect, the feeder cells and hepatocytes are of human origin.

In another aspect, provided is an in vitro co-culture system comprising feeder cells comprising growth-arrested liver non-parenchymal stromal cells, and hepatocytes; wherein the feeder cells and hepatocytes are allogenic and of mammalian origin. In a further aspect, the feeder cells and hepatocytes are of human origin.

In another aspect, provided is an in vitro co-culture system comprising feeder cells and hepatocytes, wherein the feeder cells and hepatocytes are allogeneic; wherein the feeder cells comprise growth-arrested stromal cells; and further comprising one or more additional cell populations or subpopulations (e.g., immune cells, endothelial cells, liver progenitor cells, and the like). In a further aspect, the feeder cells and hepatocytes are of human origin (e.g., isolated from humans), and the one or more additional cell populations are of an origin selected from the group consisting of human origin, or animal (i.e., mammalian) origin but which have been genetically modified to mimic human cells (“humanized”) as known to those in the art.

In another aspect, the in vitro co-culture system further comprises a structure for three-dimensional culturing comprising: a substrate or scaffold for culturing; micropatterning of a substrate and/or of the cells in the co-culture system; a bioprinted scaffold containing a component selected from cells, tissue components, artificial tissue construct, or a combination thereof; a spheroid containing cells for co-culturing in the system. The structure may further comprise channels for fluid flow (e.g. as microfluidic channels in a biochip) as known to those skilled in the art.

In another aspect, provided is a method for culturing of hepatocytes comprising contacting hepatocytes with feeder cells in an in vitro or ex vivo culture system; wherein the feeder cells and hepatocytes are allogeneic; wherein the feeder cells comprise growth-arrested stromal cells; wherein the culture system comprises a substrate for cell contact, and cell culture medium, for maintenance of viability and function of cells in the culture system; and wherein the cells in the culture system are incubated in conditions sufficient for promoting viability and function of cells in the culture system. The hepatocytes may comprise freshly isolated hepatocytes or cryopreserved hepatocytes. The stromal cells may comprise freshly isolated stromal cells or cryopreserved stromal cells, and the stromal cells may comprise liver non-parenchymal stromal cells. The stromal cells may be treated to be growth-arrested prior to cryopreservation, or following cryopreservation and during or after thawing. The method may further comprise adding to the culture system one or more cell types, populations, or subpopulations other than and in addition to hepatocytes and stromal cells. The method may further comprise a cell culture system comprising a structure for three-dimensional culturing. The method may further comprise assessing one or more markers of cells from or in the culture.

In another aspect, provided is a method of using the in vitro co-culture system to determine the activity of an agent on cells in the co-culture system, the method comprising introducing the agent into the co-culture system, and then measuring or assessing markers of one or more of function, metabolic activity, phenotype, gene expression activity, morphology, or a combination thereof, of one or more cell types contained in the co-culture system. In a further aspect, markers of the hepatocytes are assessed, or markers of the feeder cells may be assessed, or markers of one or more additional cell populations or subpopulations may be assessed, or a combination thereof. Depending on the agent and the purpose of assessing the activity of the agent, the agent may be added more than once during the period of using the co-culturing system. Similarly, assessment of activity of an agent may occur more than once during the period of co-culturing.

In another aspect, provided are a composition comprising isolated, growth-arrested stromal cells for use in co-culturing with hepatocytes resulting in cultured hepatocytes that maintain hepatocyte properties (e.g., one or more of function, phenotype, gene expression, morphology, and viability), as (for example) compared to freshly isolated primary hepatocytes, in culture for a period ranging from at least 30 days, and typically at least 40 days. In a further aspect, the stromal cells comprise liver non-parenchymal stromal cells. The stromal cells may also be isolated from the same species as the hepatocytes to be co-cultured with the stromal cells are isolated from. In a further aspect, the stromal cells are of human origin. In another aspect, the composition comprises cryopreserved, isolated, growth-arrested stromal cells.

Another aspect of the invention provides a kit comprising a composition comprising isolated stromal cells, and a composition comprising isolated hepatocytes, wherein the stromal cells and hepatocytes are isolated from the same mammalian species. In a further aspect, the stromal cells and hepatocytes are isolated from humans. The kit composition comprising isolated stromal cells may comprise growth-arrested stromal cells or, alternatively the kit composition may comprise isolated stromal cells and the kit may further comprise a composition comprising a chemical or molecule for arresting growth of the stromal cells which a user of the kit may use to treat the stromal cells to result in growth-arrested stromal cells. The isolated stromal cells in the kit may comprise cryopreserved stromal cells. The isolated stromal cells in the kit may comprise liver non-parenchymal stromal cells. The composition comprising isolated hepatocytes may comprise cryopreserved hepatocytes. The hepatocytes may be human cells. The kit may further comprise one or more compositions comprising a cell type, population, or subpopulation other than hepatocytes and stromal cells. The kit may further comprise reagents for culturing the cells (e.g., cell culture media, or nutritional components), and packaging for holding the compositions. The kit may further comprise one or more of reagents desired for characterization of cells from the kit and after subsequent co-culturing.

### BRIEF DESCRIPTION OF THE FIGURES

FIG. 1 is a graph showing basal Cytochrome p450 CYP3A4 activity in human adult hepatocytes (“Adult”) and neonatal hepatocytes (“Neonatal”), each plated alone (“Alone”) or in co-culture with growth arrested human adult non-parenchymal stromal cells (“Hep/Strom”).

FIG. 2 is a graph showing basal Cytochrome p450 CYP3A4 activity in human adult hepatocytes (“Adult”) and neonatal hepatocytes (“Neonatal”), each plated in co-culture with human growth arrested non-parenchymal stromal cells over 42 days in vitro.

FIG. 3 shows multiple graphs representing human adult hepatocytes (“Adult”) and neonatal hepatocytes (“Neonatal”) co-cultured with human growth-arrested non-parenchymal stromal cells, and measured is Cytochrome p450 CYP3A4 activity following either induction with Rifampicin (FIG. 3A-D) or inhibition with Ketoconazole (FIG. 3E-H), on days 22, 29, 36 and 43 post plating in vitro.

FIG. 4 shows photomicrographs of human adult hepatocytes plated in co-culture with growth arrested non-parenchymal stromal cells, and assessed for hepatocyte properties, including morphology and formation of bile canaliculi over 43 days of in vitro culture.

FIG. 5 shows photomicrographs of human neonatal hepatocytes plated in co-culture with growth arrested non-parenchymal stromal cells plated in co-culture with growth arrested non-parenchymal stromal cells, and assessed for hepatocyte properties, including morphology and formation of bile canaliculi over 43 days of culture.

FIG. 6 shows photomicrographs of human adult hepatocytes or neonatal hepatocytes or growth arrested non-parenchymal stromal cells plated as monocultures and assessed for viability, or ability to form canaliculi on day 10 in culture.

### DETAILED DESCRIPTION OF THE INVENTION

Described herein is a human hepatocyte/human liver stromal cell co-culture system that results in maintenance of hepatocyte properties in an in vitro culture beyond 40 days of initiation of culture, with co-cultured hepatocytes demonstrating the ability to undergo drug induction and inhibition studies, drug metabolism and cytochrome P450 activity and functional transport into bile canaliculi. In the co-culture system, the growth-arrested stromal cells provide soluble factors, extracellular matrix and cell-cell interactions necessary to nurture the hepatocytes, thereby enabling the longevity of hepatocytes in culture and the ability to do low dose long-term studies in the in vitro co-culture system that currently cannot be done using conventional hepatocyte cultures. This co-culture system also enables the use of donor lots of hepatocytes of low metabolic activity that couldn't be used for hepatocyte studies to now have value, extended utility and applications not before possible.

In one aspect, hepatocytes to be used in this co-culture system, or as a composition in a kit for the co-culture system, were isolated from the liver, cryopreserved, and then stored in liquid nitrogen freezers using standard methods in the industry prior to use in the co-culture system. The non-parenchymal stromal cells were also isolated from the liver, grown in culture in media that supports the proliferation of all the cell types in the stromal population, followed by cryopreservation, and storage in a liquid nitrogen freezer prior to use in the co-culture system. The stromal cells may be growth-arrested prior to cryopreservation, or thawed after cryopreservation and then growth-arrested using methods known in the art. The growth arrested stromal cells may be thawed concurrently with the thawing of hepatocytes to be used in co-culture. The growth-arrested stromal cells may be added to the co-culture system first, prior to adding the hepatocytes to the co-culture system. Alternatively, the growth-arrested stromal cells may be combined with the hepatocytes in cell culture medium suitable for seeding hepatocytes cultures (such medium known to those in the art and commercially available), Optimally, the growth-arrested stromal cells are combined with the hepatocytes with a range of ratios that may provide optimal support for the hepatocytes in culture to maintain hepatocyte properties. The ratios of hepatocytes to growth-arrested stromal cells for introducing into the co-culture system may range from about 1:1, 1:2, 1:3, 1:4, 1:5, or 1: to greater than 5. The substrate or structure (e.g., tissue culture plate, microtiter plate, biochip, and the like) may be coated with a coating material promoting cellular adherence (“adherence material”; e.g., protein, glycoprotein, lipid, carbohydrate, collagen, fibrinogen, fibronectin, laminin, gelatin, hyaluronic acid, a polyamine, plasma-etched polymer surface, a biomatrix comprised of decellularized liver tissue, or combination thereof). The co-culture system is then incubated in conditions sufficient for maintaining hepatocyte functions such a s viability and function (e.g., 37 degrees C., 5 to 6% CO₂). This co-culture system allows the hepatocytes to benefit from the presence of and contact with stromal cells, and prevented is stromal cells from overgrowing the co-cultured hepatocytes to the detriment of the hepatocytes. As a result of the co-culture system, hepatocytes are cultured beyond 40 days in vitro, while maintaining hepatocyte properties such as remaining physiologically functional in drug metabolism and pharmacokinetics (DMPK) assays, including absorption, distribution, metabolism, excretion and toxicity (ADMET), virology, drug-drug interaction assays, induction and inhibition assays. In addition, using this co-culture system, hepatocytes from immature donors (fetal, neonatal pediatric and adolescent) achieve mature physiological function in liver metabolic and transport activities and maintain that function in vitro beyond 40 days post plating (post-initiation of co-culture). In addition, due to the longevity of hepatocytes in this co-culture system, hepatocytes can be infected with virus such as hepatitis A, B or C, and the infected hepatocytes will begin to produce virion enabling the co-cultures system to be used to screen for anti-viral agents. Likewise, due to the longevity of hepatocytes in this co-culture system, hepatocytes remain functional in repeated assays of the same cells during the course of 40+ days in the co-culture which allows the ability to measure the metabolic and transport activity in the same population of cells over time demonstrating the ability to undergo drug induction and inhibition studies, drug metabolism and cytochrome P450 activity and functional transport into bile canaliculi in repeated assays of the same cells. This co-culture system recapitulates aspects of the native environment of the liver, and may exhibit the liver functions or hepatocyte properties including protein synthesis; formation of urea; production of serum albumin, clotting factors, enzymes, and numerous other proteins; formation and excretion of bile during bilirubin metabolism; lipid synthesis and secretion of plasma lipoproteins; regulation of carbohydrate homeostasis; control of cholesterol metabolism; storage of nutrients vitamins minerals; and metabolism or detoxification of drugs and other foreign substances. As a result, this co-culture system expands the types of assays and studies that can be done with hepatocytes in vitro or ex vivo.

Definitions—While the following terms are believed to be well understood by one of ordinary skill in the art of biotechnology, the following definitions are set forth to facilitate explanation of the invention.

The term “liver non-parenchymal stromal cells” means non-parenchymal cells isolated from a mammalian liver. The non-parenchymal cells comprise a combination of multiple cell types, populations, and subpopulation including, but not limited to, immune cells, endothelial cells, mesenchymal cells, Kupffer cells, stellate cells, and fibroblasts. Isolation of non-parenchymal stromal cells from the liver can be performed using any methods known to those skilled in the art including, but not limited to, the method illustrated in Example 1.

The term “additional cell type, population or subpopulation” that may be added to the co-culture of hepatocytes and growth-arrested stromal cells, is used herein to mean one or more of neutrophils, biliary duct cells, liver progenitor cells, sinusoidal endothelial cells, Kupffer cells, immune cells, fibroblasts, fat cells, or other cells, particularly when trying to represent a pathological state of the liver in the co-culture system. The ratio of hepatocytes to additional cell types, population and subpopulations will vary depending on the additional cells to be added, but may range from 1:0.5 to 1:0.005. state).

The term “growth-arrested” when used herein in referring to stromal cells or liver non-parenchymal stromal cells, refers to such cells after treatment with a molecule or composition that is capable of inhibiting growth of such treated cells while still preserving substantial viability and function of the treated cells, as compared to cells not treated with a growth inhibitor. Illustrative examples of molecules or compositions are known to those skilled in the art, and may include, but are not limited to, mitomycin C, trichostatin A, colchicine, Epothilone B, MPC6827, Pladienolide B, diadzein, Plumbagin and CPI203.

The term “agent” is used herein to refer to a substance, composition, or organism to be assessed for its activity on cells in the co-culture system, and particularly effects on hepatocytes, means one or more of a hormone, an infectious agent (such as a virus that infects hepatocytes, including, but not limited to Hepatitis A virus, Hepatitis B virus, and Hepatitis C virus, any virus that can infect the liver, protein, lipid, liposome, vesicle, cytotoxin, drug, biologic, growth factor, cytokine, consumer product, nanoparticle, carbohydrate, nutraceutical, natural product (one or more herbs, herb extracts), nutritional product, pharmaceutical, and chemical.

The term “maintain hepatocyte properties” refers to maintenance of hepatocyte function. Hepatocyte function may include, but not be limited to, one or more of metabolic and transport activity, albumin secretion, cytochrome P450 activity, urea synthesis, carbohydrate and lipid metabolism, production of blood clotting factors, detoxification. Maintaining function may be represented by the hepatocytes in the co-culture demonstrating at least 50%, at least 60%, at least 70%, or at least 80% of the same measured hepatocyte function demonstrated by freshly isolated hepatocytes. Illustrative methods for measuring hepatocyte function can be performed by methods known in the art to include metabolic assays, transporter assays, immunoassays, polymerase chain reaction (for measuring gene expression), and high content imaging.

The present invention will be described in the following examples, which are illustrative in nature.

### EXAMPLE 1

In this example, illustrated is isolating and processing of liver non-parenchymal stromal cells, including growth-arrested stromal cells. Whole or resected livers were obtained through informed consent, and processed using standard methods known in the art for enzymatic digestion of the liver tissue resulting in a liver cell suspension. The liver cell suspension was further processed using a colloidal medium (e.g., Percoll) for a gradient centrifugation to isolate the parenchymal hepatocytes. The remaining cell suspension, comprising non-parenchymal stromal cells (“liver stromal cells”), was pelleted by centrifugation at 500g for 5 minutes at 4 degrees C. The resultant cell pellet was resuspended in a suitable commercially available cell culture medium (e.g., MSC Growth Medium; PhoenixSongs) further supplemented with 20ng/ml epidermal growth factor (EGF) and 0.1 mM beta mercaptoethanol, the cells were then plated in T225 tissue culture flasks at 10.0×10⁶ cells per flask and then placed in a humidified incubator with 6% CO₂. The liver stromal cells were fed with a complete media change the next day, and every other day thereafter while in culture.

When the liver stromal cells reached 85-90% confluency, the cells were dissociated with a commercially available cell dissociation reagent (ACCUTASE). For example, cell culture media was removed from the liver stromal cells, and 15 ml cell dissociation reagent was added to the cells in the cell culture flask for 10-15 minutes at room temperature. The dissociated cells were then transferred into a tube for centrifugation at 500 g for 5 minutes at 4 degrees C. The resultant cell pellet was then resuspended in cell culture medium. If in the processing there were multiple pellets, all the pellets were pooled into one cell suspension. An aliquot was then taken to determine cell count and viability. The liver stromal cells were then plated in T225 tissue culture flasks at a density of 3×10⁶ cells/flask and returned to the humidified incubator. This process can be repeated for continued expansion and cryopreservation of liver stromal cells.

In this example, illustrated is cryopreservation of the liver stromal cells. From the pooled dissociated liver stromal cells, an aliquot was removed to determine cell count and viability. The pooled cell suspension was pelleted again by centrifugation at 500 g for 5 minutes at 4 degrees C. The liver stromal cell pellet was resuspended in commercially available freezing medium at a cell density of 9×10⁶ cells/mL, and 1 ml per vial is aliquoted for cryopreservation as a master bank. Frozen vials were stored in a liquid nitrogen freezer.

In this example, illustrated is treating liver stromal cells to become growth arrested. One vial from the master bank was thawed into cell culture medium and pelleted by centrifugation at 500 g for 5 minutes at 4 degrees C. The pellet was resuspended in the cell culture medium and plated into 3 T225 Flasks that were then transferred into a humidified incubator at 37 degrees C. in 6% CO². When the liver stromal cells reached 85-90% confluency, the cells were dissociated as described above in this example. When the liver stromal cells were expanded to approximately 5-10×10⁸, the liver stromal cells were growth arrested. For example, when the liver stromal cells were 100% confluent, the cells were growth arrested by incubation for 2 hours at 37 degrees C. in cell culture medium supplemented with a growth inhibitor (e.g., 10 ug/ml mitomicin C). The medium supplemented with the growth medium was removed, and the liver stromal cells were dissociated and then cryopreserved as described previously in this example. Growth arrested liver stromal cells were cryopreserved at 5×10⁶ cells/mL and 1 mL per vial. Cryopreserved growth arrested liver stromal cells were stored in liquid nitrogen freezer.

### EXAMPLE 2

Illustrated in this Example are compositions and methods for co-culturing hepatocytes and growth-arrested liver stromal cells. One vial of cryopreserved hepatocytes was thawed into commercially available hepatocyte recovery medium, and one vial of growth arrested liver stromal cells was thawed into commercially available hepatocyte plating medium (“cell culture medium”). Each of these cell suspensions were centrifuged to pellet the cells (hepatocytes at 100 g, liver stromal cells at 500 g, both at 4 degrees C. for 5 minutes) to remove the cryoprotectant media. Each cell pellet was resuspended in cell culture medium. For comparison purposes, fresh hepatocytes in suspension were centrifuged to pellet the cells at 100 g at 4 degrees C. for 5 minutes to remove transport medium. The fresh hepatocytes were resuspended in cell culture medium. The hepatocytes (fresh or cryopreserved) were combined with the growth-arrested liver stromal cell suspensions at an optimal ratio (illustrated here is a 1:1 ratio). The pooled hepatocyte and growth-arrested liver stromal cell suspension is then plated at an optimal density for the tissue culture plate (see Table 1) that has been coated with an adherence material. The pooled hepatocyte and growth-arrested liver stromal cell suspension may be plated into any plate format that has been coated with the desired adherence material required for specific metabolic or transport assays. For example, the day following plating of the pooled hepatocyte and growth arrested liver stromal cell population, the cell culture medium is refreshed with a complete media change by removing the cell culture medium and replacing it with a commercially available cell culture medium for hepatocyte maintenance (PhoenixSongs Cat. #: CMM-250 or CMM500). During the time the cells remain in co-culture (40+ days), the hepatocytes and liver stromal cell in the co-culture appear healthy and functional when the cell culture medium is refreshed daily by removing the medium from the cells and replacing it with fresh cell culture medium.

TABLE 1
Plating densities for human hepatocytes and
human liver stromal cells at a 1:1 ratio.

| Dish/Flask | Growth Area | Media Volume | Hepatocytes | Stromal Cells |
|---|---|---|---|---|
| Size | (cm²) | (ml) per well | per well | per well |
| 6-Well | 9.6 | 2 | 1.44 × 10⁶ | 1.44 × 10⁶ |
| 12-Well | 3.8 | 1 | 5.70 × 10⁵ | 5.70 × 10⁵ |
| 24-Well | 2.0 | 0.5 | 3.00 × 10⁵ | 3.00 × 10⁵ |
| 48-Well | 1.1 | 0.2 | 1.65 × 10⁵ | 1.65 × 10⁵ |
| 96-Well | .32 | 0.1 | 4.80 × 10⁴ | 4.80 × 10⁴ |
| 384-Well | .136 | 0.05 | 2.04 × 10⁴ | 2.04 × 10⁴ |

### EXAMPLE 3

Illustrated in this Example is the use of compositions of co-culturing hepatocytes and growth-arrested liver stromal cells on hepatocyte metabolic functions and results. Human adult hepatocytes or neonatal hepatocytes were plated alone or together in co-culture with growth arrested human non-parenchymal stromal cells in commercially available cell culture medium on collagen I coated 96-well plates to compare traditional hepatocyte culturing methods to this co-culture method. The next day after plating, and every day thereafter, the medium was removed and replaced with fresh medium. Functionality of the hepatocytes was used to compare hepatocytes plated alone to hepatocytes plated together with growth arrested non-parenchymal liver stromal cells. As a representative measure of maintenance of hepatocyte properties, measured was cytochrome P450 CYP3A4 activity. A commercially available luminescence CYP3A4/Luciferin-IPA biochemical assay was used in 96-well format to measure basal cytochrome P450 CYP3A4 basal activity. From the same preparation of hepatocytes, a sample was plated alone, and another sample was plated together with growth arrested human liver stromal cells. The assay was carried out on days 1, 5, 15, 19 and 25 post plating. On the day of the assay, the media was removed and replaced with 60 μL of cell culture medium containing the CYP3A4/Luciferin-IPA substrate, and the plate was returned to the humidified incubator for the reaction to continue for 1 hour at 37 degrees C. in 6% CO₂. Following incubation, 50 μL of the reaction was transferred into the white luminescence assay plate, and 50pL of the luciferin detection solution was added to each well in the assay plate. The reaction was carried out for 20 minutes in the dark, and then the plate was placed into the luminometer. The CYP3A4 metabolic enzyme inside the hepatocytes will metabolize the luminogenic P$50 CYP3A4/Luciferin-IPA substrate and produce a luciferin metabolite that will release light in the reaction with the luciferin detection reagent that can be detected by a luminometer. The amount of light directly correlates with the amount of CYP3A4 activity inside the hepatocytes.

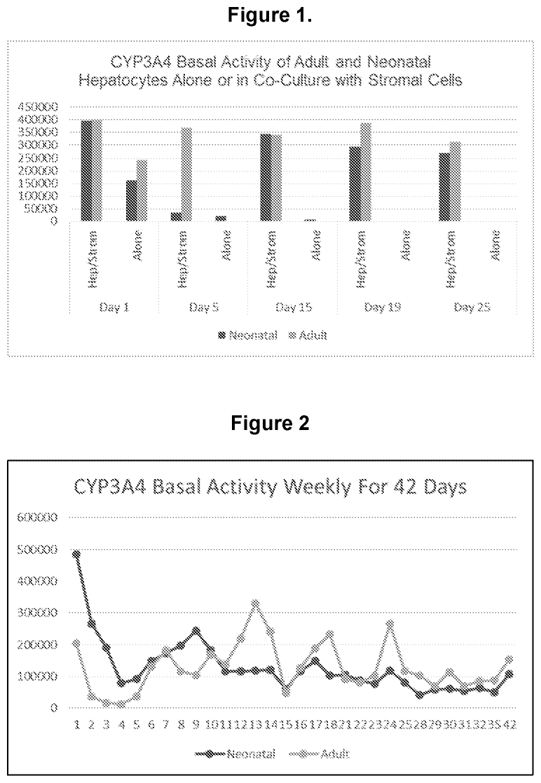

As shown in FIG. 1, CYP3A4 basal activity was maintained in both adult human hepatocytes in co-culture with growth arrested human liver stromal cells and neonatal human hepatocytes in co-culture with growth arrested human stromal cells throughout the 25 days in vitro. In contrast, adult human hepatocytes and neonatal human hepatocytes cultured without liver stromal cells showed a rapid decline of CYP3A4 basal activity, wherein activity was not measurable on day 5 for the adult hepatocytes, and only a minimal activity was detected on day 5 in the neonatal hepatocytes (FIG. 1). By day 5, many hepatocytes cultured without the liver stromal cells were dying.

### EXAMPLE 4

Illustrated in this Example is the use of compositions of co-culturing hepatocytes with growth-arrested liver stromal cells in repeated assays to demonstrate hepatocyte functions and results. Adult hepatocytes and neonatal human hepatocytes were each plated in co-culture with growth arrested human liver stromal cells as described above, and at a density of 4.8×10⁴ cells/well. The next day and every day thereafter the media was removed and replaced with fresh commercially available cell culture medium. Cytochrome P450 CYP3A4 activity in the human hepatocytes was measured as described above. The assay was repeated daily on the same cells for 42 days post plating to demonstrate that human hepatocytes in co-culture with growth arrested human stromal cells could undergo daily assays on the same cells for over 40 days post plating and maintain hepatocyte properties such as metabolic functions. For example, each day of the assay, the media was removed and replaced with 60pL of cell culture medium containing the CYP3A4/Luciferin-IPA substrate and the plate was returned to the humidified incubator for the reaction to continue for 1 hour at 37 degrees C. in 6% CO₂. Following incubation, 50 μL of the reaction was transferred into the white luminescence assay plate and 50 μL of the luciferin detection solution was added to each well in the assay plate. The reaction was carried out for 20 minutes in the dark and then the plate is placed into the luminometer. As shown in FIG. 2, CPY3A4 basal metabolic activity was sustained in in both adult hepatocytes in co-culture with growth arrested human liver stromal cells and neonatal human hepatocytes in co-culture with growth arrested human stromal cells over the 42 days of repeated assays. Thus, maintenance of hepatocyte properties was demonstrated (e.g., maintained was metabolic activity) over the course of 42 days in vitro as measured by CYP3A4 basal activity.

### EXAMPLE 5

Illustrated in this Example is the use of compositions of co-culturing hepatocytes with growth-arrested liver stromal cells in repeated assays to demonstrate hepatocyte reaction to chemical agents for the induction of cytochrome P450 CYP3A4 activity and chemical agents for the inhibition of cytochrome P450 CYP3A4 activity. Shown in FIGS.

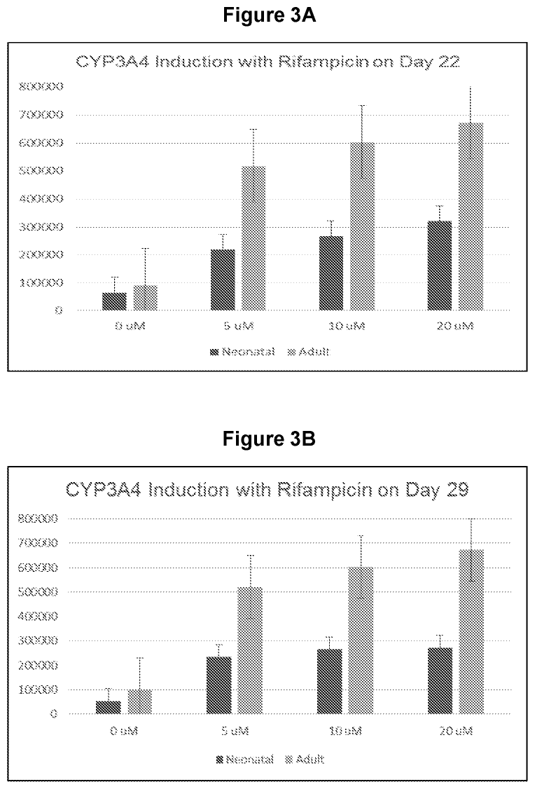

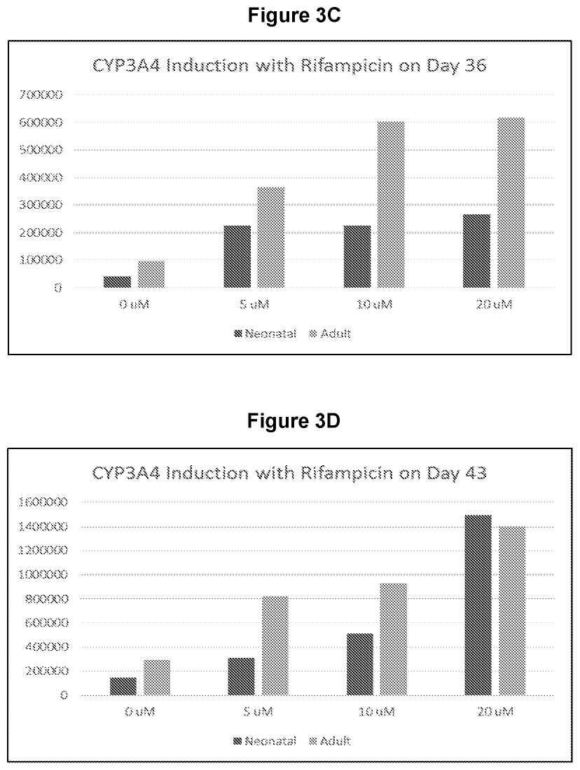

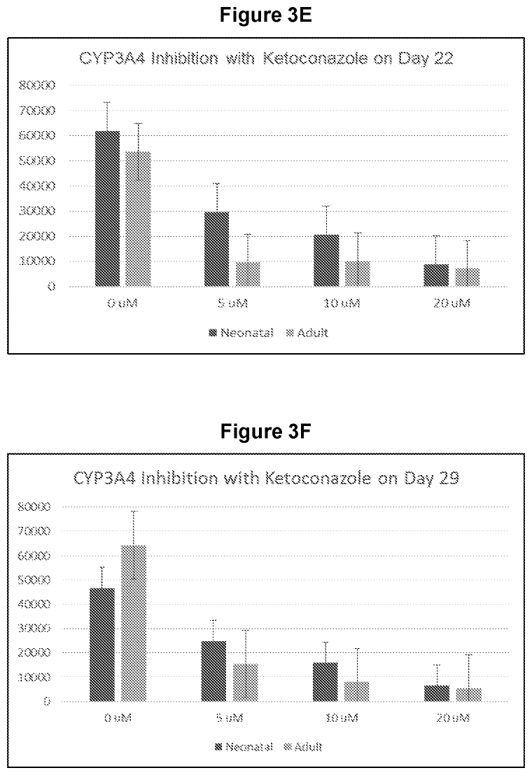

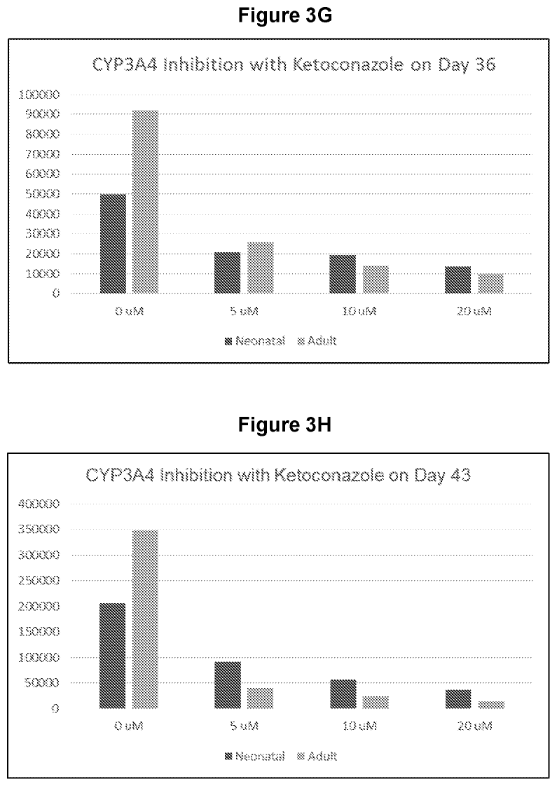

3A-D is a dose dependent response of Cytochrome p450 CYP3A4 induction with Rifampicin at 0, 5, 10 and 20 μM concentrations in cell culture medium for 24 hours followed by CYP3A4 activity measured using a luminescence assay on days 22 (FIG. 3A), 29 (FIG. 3B), 36 (FIG. 3C) and 43 (FIG. 3D) in co-culturing in vitro. As shown in FIGS. 3A-D, there was an average of 6.5-fold induction of CYP3A4 activity in adult hepatocytes and an average of 5.75-fold induction of CYP3A4 activity in neonatal hepatocytes, when comparing the activity of hepatocytes receiving 0 μM to the activity of hepatocytes receiving 5, 10, or 20 μM of Rifampicin.

Shown in FIGS. 3E-H is a dose dependent response of Cytochrome p450 CYP3A4 inhibition with Ketoconazole at 0, 5, 10 and 20 μM concentrations in cell culture medium for 24 hours followed by CYP3A4 activity measured using a luminescence assay on days 22 (FIG. 3E), 29 (FIG. 3F), 36 (FIG. 3G) and 43 (FIG. 3H) in co-culturing in vitro. As shown in FIGS. 3E-H, there was an average of 10-fold inhibition of CYP3A4 activity in adult hepatocytes and an average of 5.75-fold inhibition in neonatal hepatocytes following administration of Ketoconazole (5, 10, or 20 μM) to the hepatocytes. Thus, the longevity and maintenance of hepatocytes in this co-culture allows repeated long-term testing of hepatocytes to exogenous agents,

### EXAMPLE 6

Illustrated in this Example is the use of compositions of co-culturing hepatocytes with growth-arrested liver stromal cells in repeated assays to demonstrate hepatocyte transport functions, longevity in culture and results. In this example, human adult hepatocytes and neonatal hepatocytes were each plated in co-culture with growth arrested human liver stromal cells on collagen I coated 24-well plates, and the same cells were repeatedly assayed weekly to demonstrate maintenance of hepatocyte properties comprising functional transport into bile canaliculi. Human adult hepatocytes or neonatal hepatocytes were co-cultured with growth arrested liver stromal cells, and compared to adult hepatocytes or neonatal hepatocytes (as a control for cell viability and longevity) or the growth-arrested liver stromal cells (as a control, for example, to demonstrate that the stromal cells don't metabolize CDFDA so there is no transport of CDF). On the day of the assay, the cell culture medium was removed and replaced with medium containing 5 μM 5 (and 6)-carboxy-2′,7′-dichlorofluorescein diacetate (CDFDA) and then put into a humidified incubator at 37 degrees C. and 6% CO₂ for 20 minutes. Following incubation, the cells were washed 3 times with Hanks balanced salt solution (HBSS) to remove the CDFDA that remained in the media, and added was fresh medium.

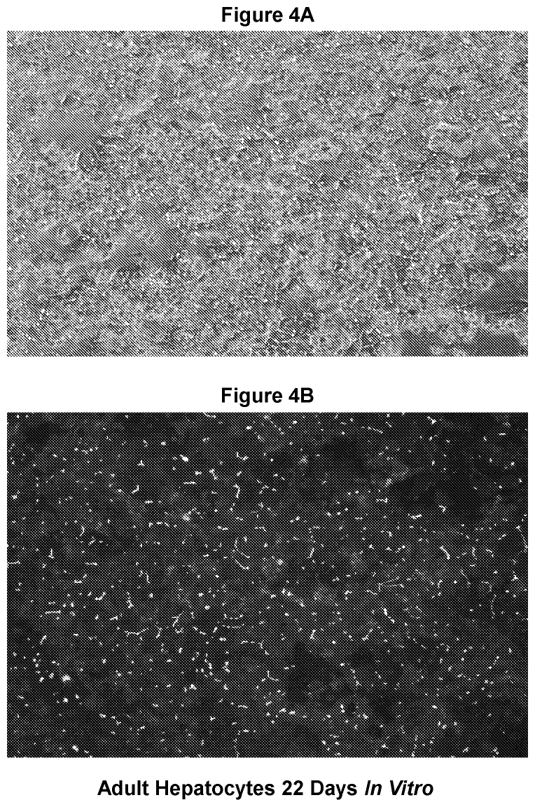

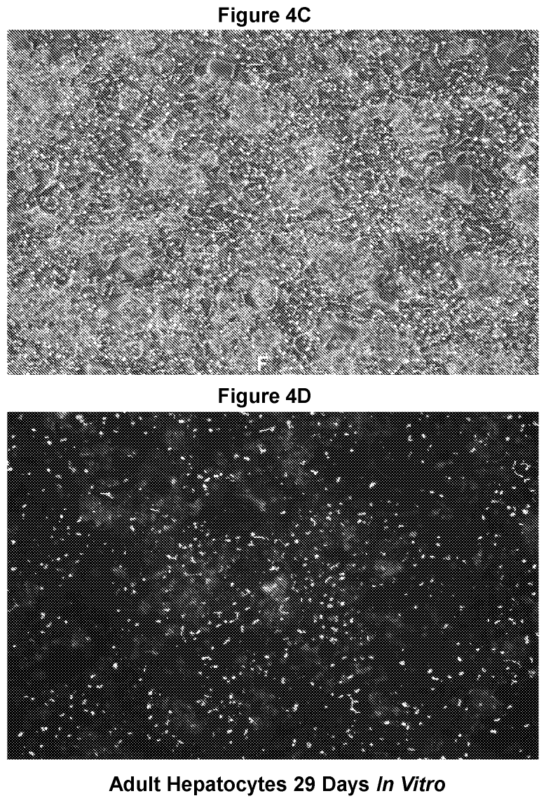

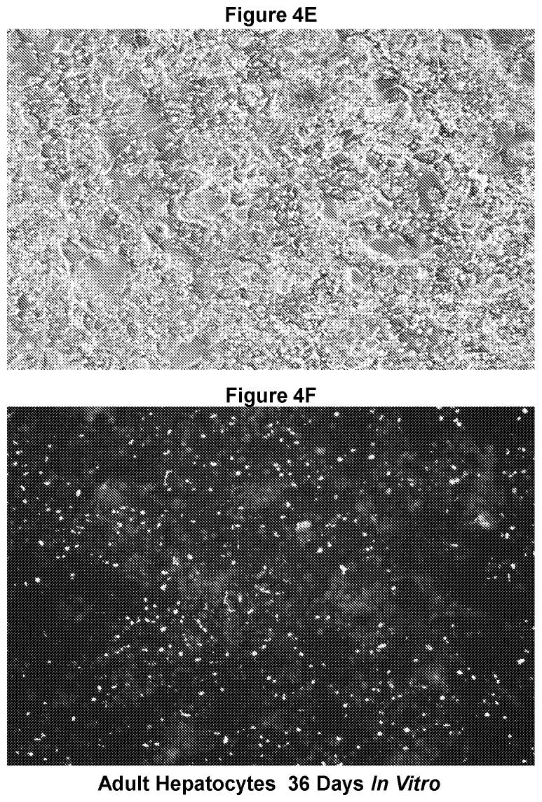

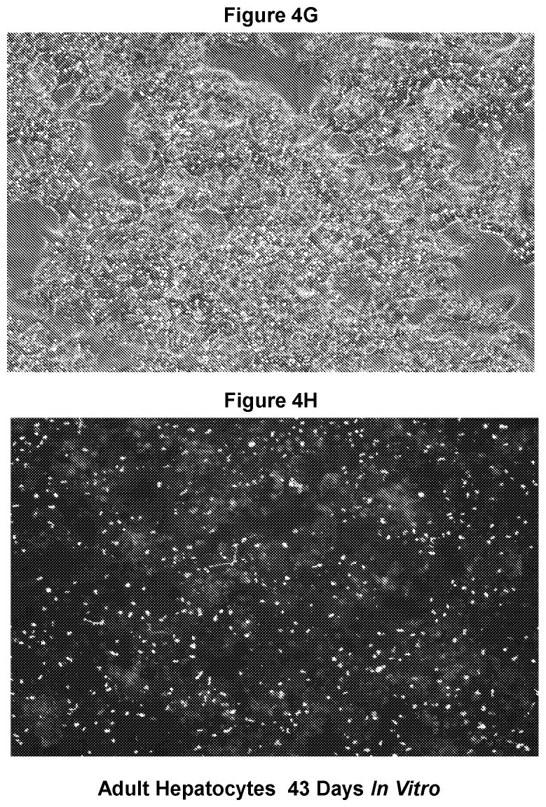

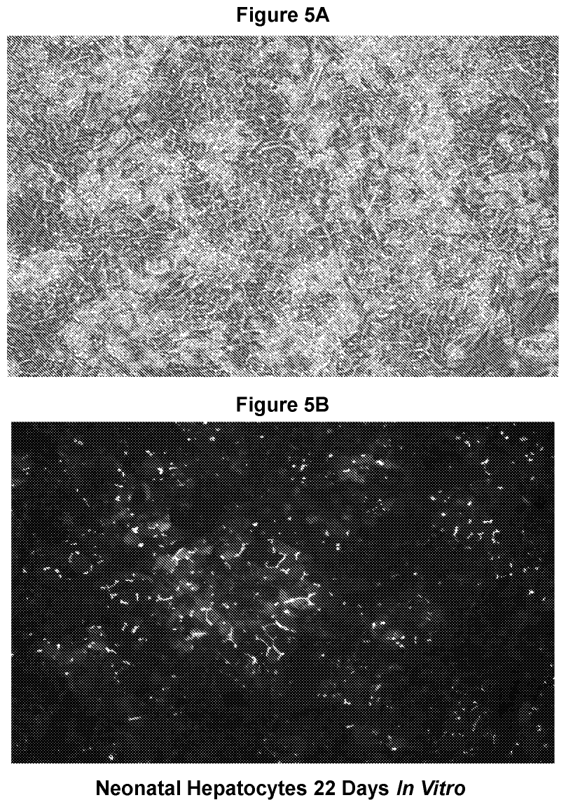

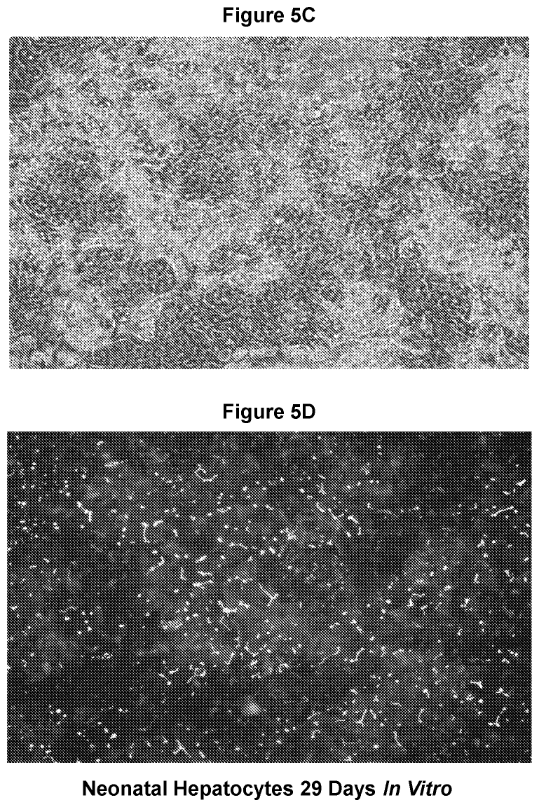

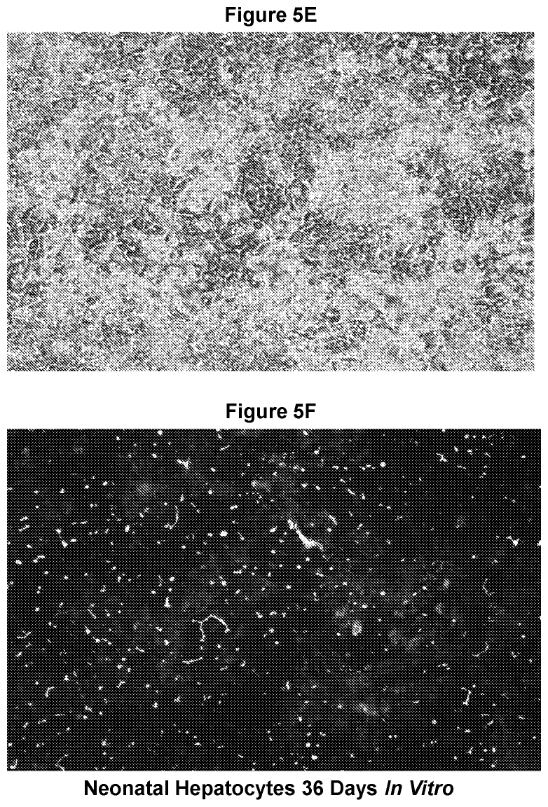

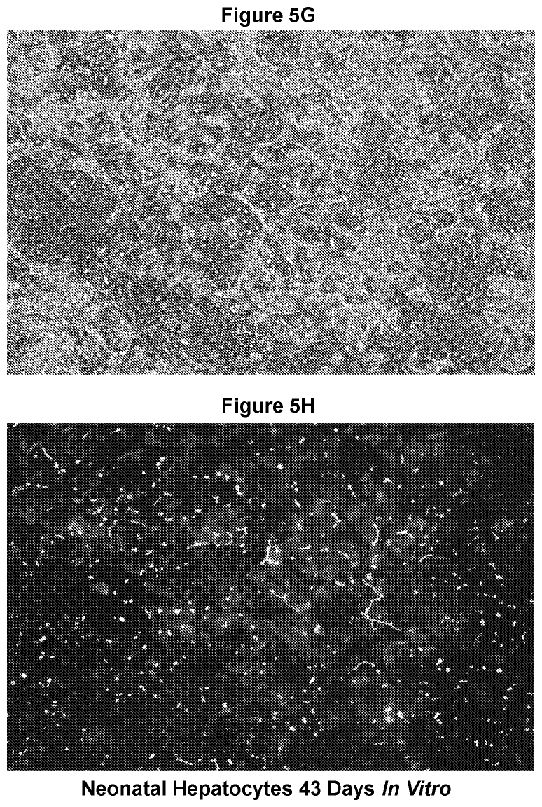

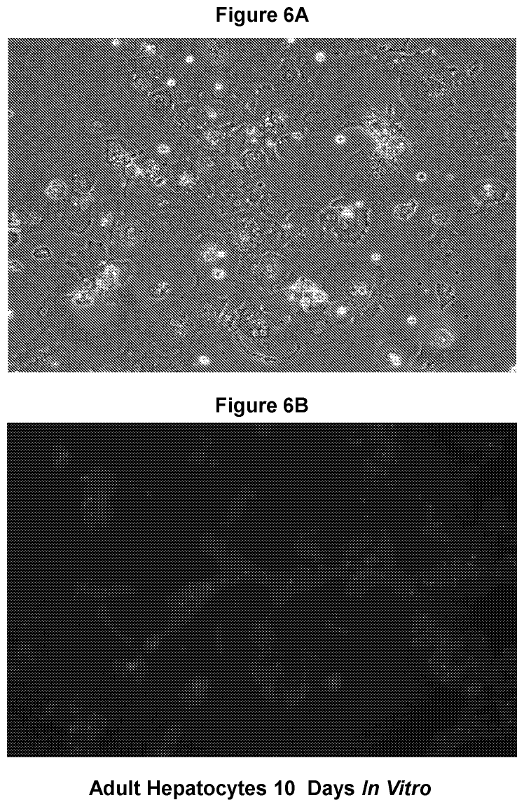

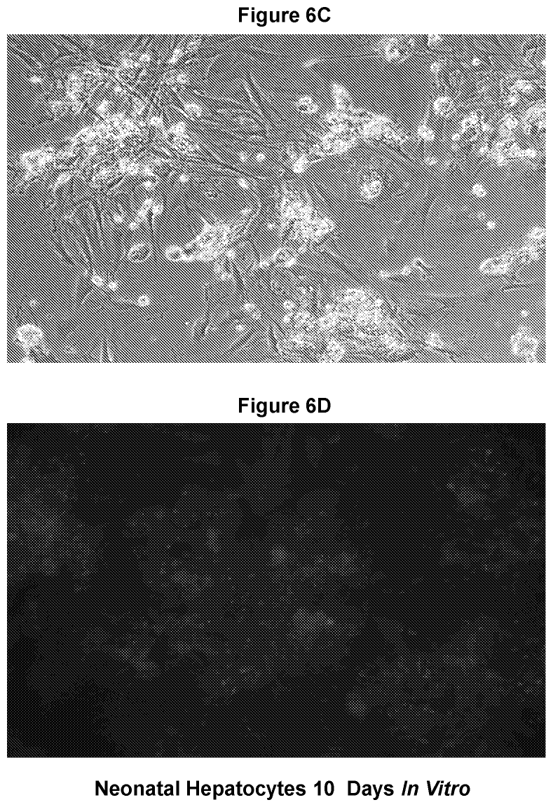

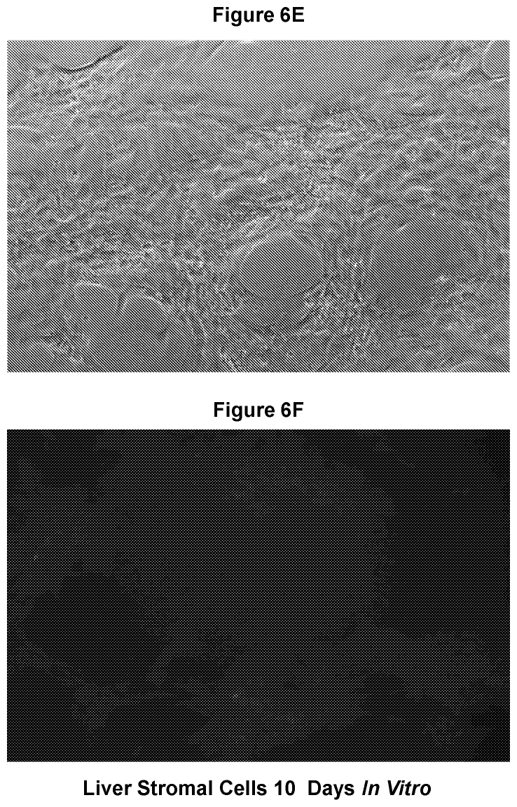

In this example, the CDFDA diffuses into hepatocytes where it is hydrolyzed to fluorescent 5 (and 6)-carboxy-2′,7′-dichlorofluorescein (CDF) inside the hepatocytes, and then transported from the hepatocytes into bile canaliculi by MRP2 (a bile acid transporter). To visualize the CDF in the canaliculi, the cells were examined microscopically with epifluorescence Alexa 488 filter set. Photomicrographs were taken at 100× magnification to detect fluorescent canaliculi which indicates that the metabolized CDFDA was hydrolyzed to CDF and transported from the hepatocytes into the canaliculi. As shown in FIG. 4, phase micrographs (A, C, E, G), on days 22, 29, 36 and 43 demonstrated is adult hepatocyte morphology characterized by healthy looking cuboidal shaped hepatocytes, tightly connected with distinct borders between adjacent cells known as canaliculi, suggesting that these hepatocytes are polarized in demonstrating the morphology of parenchymal cells in the liver. Fluorescent photomicrographs (B, D, F, H) on days 22, 29, 36 and 43 demonstrated (see fluorescence) was development of bile canaliculi which appear as white fluorescent markings in the black background of the fluorescent micrograph. As shown in FIG. 5, phase micrographs (A, C, E, G), on days 22, 29, 36 and 43 demonstrated is neonatal hepatocytes matured in vitro to mature hepatocyte morphology characterized by healthy looking cuboidal shaped hepatocytes, tightly connected with distinct borders between adjacent cells known as canaliculi, suggesting that these hepatocytes are polarized in demonstrating the morphology of parenchymal cells in the liver and fluorescent photomicrographs (B, D, F, H) on days 22, 29, 36 and 43 demonstrated (see fluorescence) was development of bile canaliculi which appear as white fluorescent markings in the black background of the fluorescent micrograph. As shown in FIG. 6, when the adult hepatocytes (A) or neonatal hepatocytes (C) were plated alone, they lose hepatocyte morphology and take on more of a mesenchymal morphology indicating that the hepatocytes had undergone the epithelial to mesenchymal transition causing the hepatocytes to loose polarity, hence hepatocyte function and acquire the motility of mesenchymal cells which is typical of hepatocytes in culture alone. Growth arrested stromal cells (E) remain healthy and cluster which is typical of stromal morphology. There is no fluorescence appearing in adult hepatocytes (B) or in neonatal hepatocytes (D) demonstrating that there were no fluorescent canaliculi formed in the hepatocytes plated alone by day 10 post plating; and the liver stromal cells plated alone (F) don't metabolize the CDFDA as is illustrated by lack of fluorescent canaliculi.

What is claimed is:

1. A method for culturing of hepatocytes comprising contacting hepatocytes with feeder cells in an in vitro or ex vivo culture system; wherein the feeder cells and hepatocytes are isolated from the same species; wherein the feeder cells comprise growth-arrested stromal cells; wherein the culture system comprises a substrate for cell contact, and cell culture medium; and wherein the cells in the culture system are incubated in conditions sufficient for promoting viability and function of cells in the culture system.

2. The method of claim 1, wherein the hepatocytes are human cells, and the feeder cells comprise liver non-parenchymal stromal cells that are human cells.

3. The method of claim 1, wherein the hepatocytes cultured in the culture system maintain hepatocyte function for at least 30 days.

4. An in vitro co-culture system comprising feeder cells comprising growth-arrested liver non-parenchymal stromal cells, and hepatocytes; wherein the feeder cells and hepatocytes of human origin; wherein the hepatocytes are contacted with the feeder cells;
and wherein the co-culture system further comprises a substrate for cell contact, and cell culture medium.

5. The co-culture system of claim 4, further comprising the substrate further comprising adherence material.

6. The co-culture system of claim 4, further comprising the substrate further comprising a structure for three-dimensional culturing.

7. A kit comprising a composition comprising isolated stromal cells, and a composition comprising isolated hepatocytes, wherein the stromal cells and hepatocytes are isolated from the same mammalian species, and the stromal cells comprise growth-arrested stromal cells.

8. A Kit comprising a composition comprising hepatocytes and growth arrested stromal cells and a composition comprising Hepatocyte Recovery Medium and a composition comprising Hepatocyte Plating Medium and a composition comprising Hepatocyte Maintenance Medium.
